(19) United States
(12) Patent Application Publication
(10) Pub. No.: US 2019/0343457 A1
(43) Pub. Date: Nov. 14, 2019

(54) PAIN ASSESSMENT METHOD AND APPARATUS FOR PATIENTS UNABLE TO SELF-REPORT PAIN

(71) Applicant: THE REGENTS OF THE UNIVERSITY OF CALIFORNIA, Oakland, CA (US)

(72) Inventors: Amir M. Rahmani, Irvine, CA (US); Nikil Dutt, Irvine, CA (US); Kai Zheng, Irvine, CA (US); Ariana Nelson, Orange, CA (US); Pasi Liljeberg, Turku (FI); Sanna Salantera, Turku (FI); Mingzhe Jiang, Turku (FI); Arman Anzanpour, Turku (FI); Elise Syrjala, Turku (FI); Riitta Mieronkoski, Turku (FI); Geng Yang, Hangzhou (CN)

(21) Appl. No.: 16/406,739

(22) Filed: May 8, 2019

Related U.S. Application Data
(60) Provisional application No. 62/668,712, filed on May 8, 2018.

(51) Int. Cl. A61B 5/00; G16H 50/30; G06N 3/08; A61B 5/0492; A61B 5/0205

(52) U.S. Cl. CPC A61B 5/4824; G16H 50/30; G06N 3/08; A61B 5/0492; A61B 5/02416; A61B 5/0205; A61B 5/0002; A61B 5/7264; A61B 5/6803

(57) ABSTRACT

Systems and methods for automatic pain monitoring and assessment are described herein. In one example, the system may include a wearable facial expression capturing system that is placed over a subject's face. The system may be embedded with a plurality of sensors configured to detect biosignals from facial muscles and may additionally include a sensor node that recognizes facial expressions based on the detected biosignals. Pain experienced by the subject is assessed based on the facial expressions in conjunction with physiological signals obtained by other wearable sensors.

### CROSS-REFERENCES TO RELATED APPLICATIONS

This application is a non-provisional and claims benefit of U.S. Patent Application No. 62/668,712 filed May 8, 2018, the specification(s) of which is/are incorporated herein in their entirety by reference.

### GOVERNMENT SUPPORT

This invention was made with government support under Grant No./Funding Decision No. 286915 awarded by Academy of Finland. The government has certain rights in the invention.

### FIELD OF THE INVENTION

The present invention relates to systems and method for pain assessment and continuous monitoring of pain in patients, more specifically in patients who are unable to report pain.

### BACKGROUND OF THE INVENTION

Currently there is no way to objectively assess patients' pain especially from patients that have difficulties to communicate. We developed and tested a method and a smart tool that assesses pain by utilizing physiological parameters monitored by wearable devices. Although pain is believed to be an individual sensation relying on subjective assessment, objective assessment tool is needed for the wellbeing and improved care processes of noncommunicative patients. Such a tool also benefits other patient populations with more accurate medication and clinical-assisted treatment.

Pain is a severe problem for almost all patient groups. It is especially challenging for patients who can't self report their experience. Pain remains poorly managed partly because it is not recognized and assessed properly. In pain assessment, self-report is conventionally considered as the “gold standard”, which needs patients to answer questions verbally, in writing, with finger span or blinking eyes to yes or no questions. In the self-report method, the pain intensity is reported by the patient as numeric scales, which is based on two prerequisites: the patient's cognitive competence and unbiased communication. Although taken as the “gold standard”, this unidimensional model is questioned and debated for its oversimplification and limitation in several vulnerable patient populations. In practice, however, a broader range of non-self-report resources are observed by experienced clinicians to assess pain, for example, grimacing facial expression and body movements as behavioral observation and vital signs as physiologic monitoring. These non-self-report strategies are the theoretical basis and inspiration in developing an automatic pain assessment method to assist and even to replace the subjective self-report method.

In the past several decades, researchers and scientists have been trying to decode pain by monitoring electrical biosignals in different patient populations with a certain type of pain. So far, some correlation is found between electrical biosignals and pain but no individual one is sufficient enough to indicate the presence of pain due to the complexity of automatic nervous system and pain expression. As a consequence, alternative comprehensive model of pain from multiple electrical biosignals are explored. Existing models built in last five years mainly involve physiological pain indicators only from either healthy volunteers with a single type of experimental pain or patients in surgery and few has been applied in a different database for model validation. Furthermore, no model has yet been developed into an automatic pain assessment tool.

### SUMMARY OF THE INVENTION

Conventional self-report has been taken as the “gold standard” in clinical pain assessment, which need answers from patients to questions or a questionnaire verbally, in writing, with finger span or blinking eyes to yes or no questions. However, there are patients unable to self-report due to cognitive, developmental, or physiologic issues, for example, preverbal toddlers and critically ill patients. The present invention discloses a precise and automatic tool for pain assessment by biosignals acquisition and analysis with wearable sensor device. Through monitoring behavioral and physiological signs, the appearance of pain and pain state are continuously tracked. The present invention additionally discloses the design of a wearable facial expression capture system and a data fusion method.

The present invention further provides automatic and continuous monitoring of pain intensity in patients who are otherwise unable to self-report. The real-time information of the continuous monitoring can be updated to a caregiver nearby or even in a remote location, so as to improve the nursing efficiency and optimize pain management in medication. The present invention includes a multi modal integration of a plurality of physiological and behavioral signals to accurately estimate the pain experienced by the patient. Compared with a single monitoring of physiological signals or behavioral signals, a fusion or integration of the two potential pain indicators contributes a more multidimensional and comprehensive model in automatic pain assessment. In addition, the integration of wearable devices ensures the long term monitoring in patients with lightweight and portable equipment.

In some aspects, the present invention features a facial expression capturing system for measuring pain levels experienced by a human. The system may comprise a flexible mask contoured to at least partially cover one side of the human's face, the mask having an eye recess or opening disposed between an elongated forehead portion of the mask, which is above the eye recess, and a cheek portion of the mask, which is beneath the eye recess; six sensor positions located on the mask such that two sensor positions are located laterally on the elongated forehead portion of the mask and the other four sensor positions located on the cheek portion of the mask and situated in a 2 by 2 arrangement; two or more sensors embedded in the mask, wherein each sensor occupies one of the sensor positions; a sensor node disposed on a lateral flap extending from the cheek portion of the mask, wherein the sensor node comprises a processing module and a transmitter; and connecting leads electrically coupling each of the two or more sensors to the sensor node. When the flexible mask is applied to partially cover one side of the human's face, the sensor positions align with pain-related facial muscles in the human's face and the sensors are configured to detect biosignals from underlying facial muscles such as, for example, the frontalis, corrugator, orbicularis oculi, levator, zygomaticus, and risorius. In some embodiments, the processing module is configured to (i) receive the biosignals from the plurality of sensors, (ii) analyze the biosignals to deduce facial expressions and monitor pain intensity levels experienced by the subject based on the deduced facial expressions, and (iii) transmit the pain intensity levels to a medical care provider, thus allowing the medical care provider to continually monitor the pain intensity levels experienced by the subject thereby providing effective and efficient pain management.

In some embodiments, the flexible mask is composed of polydimethyl silicone elastomer (PDMS). In other embodiments, the sensors (104) comprise Ag/AgCl electrodes. The electrodes may be disposed on an inner surface of the mask (102) such that the electrodes are directly contacting skin when the mask is placed on the human's face.

In one embodiment, the system may include two sensors, where a first sensor occupies a distal-most sensor position located on the forehead portion of the mask, and a second sensor occupies a first row and first column of the 2 by 2 arrangement in the cheek portion of the mask. In a preferred embodiment, the first sensor can detect biosignals from a corrugator facial muscle and the second sensor can detect biosignals from a zygomatic facial muscle.

In another embodiment, the system may comprise five sensors, where a first sensor and a second sensor occupy the two sensor positions on the forehead portion of the mask, a third sensor and a fourth sensor occupy the sensor positions at a first row of the 2 by 2 arrangement in the cheek portion of the mask, and a fifth sensor occupies the sensor position at a second row and second column of the 2 by 2 arrangement. The first sensor can detect biosignals from a corrugator facial muscle, the second sensor can detect biosignals from a frontalis facial muscle, the third sensor can detect biosignals from a levator facial muscle, the fourth sensor can detect biosignals from an orbicularis oculi facial muscle, and the fifth sensor can detect biosignals from a zygomatic facial muscle.

In other aspects, the present invention provides a method for integrating surface electromyogram (sEMG) signals and physiological signals for automatically detecting pain intensity levels experienced by a human. One embodiment of the method may comprise providing a wearable facial expression capturing system for measuring said pain intensity levels. The system includes a flexible mask contoured to at least partially cover one side of the human's face, the mask having an eye recess or opening disposed between an elongated forehead portion of the mask, which is above the eye recess, and a cheek portion of the mask, which is beneath the eye recess; at least two sensors disposed in the mask, wherein a first sensor is disposed in the forehead portion of the mask, and a second sensor is disposed in the cheek portion of the mask; a sensor node disposed on a lateral flap extending from the cheek portion of the mask, the sensor node comprising a processing module and a transmitter; and connecting leads electrically coupling each of the at least two sensors to the sensor node.

The method further comprises applying the flexible mask to partially cover one side of the human's face such that the first sensor aligns with a corrugator facial muscle and the second sensor aligns with a zygomatic facial muscle, detecting sEMG signals from the corrugator facial muscle and the zygomatic facial muscle via the first and second sensors, respectively, filtering the detected sEMG signals via the processing module, transmitting the filtered sEMG signals to a data processing system via the wireless transmitter, and receiving physiological signals transmitted from one or more wearable sensors to the data processing system. In some embodiments, the physiological signals may comprise one or more of a breath rate, a heart rate, a galvanic skin response (GSR), or a photoplethysmogram (PPG) signal. The method continues with extracting features from each of the sEMG signals and the physiological signals, performing feature alignment on features extracted from the sEMG signals and the physiological signals, performing interindividual standardization on each of the sEMG signals and the physiological signals, performing pattern recognition by comparing the sEMG signals and the physiological signals to a database, correlating patterns recognized with pain intensity levels and classifying the pain intensity levels, and displaying the pain intensity levels to a medical care provider, thus allowing for continuous and automatic pain monitoring.

In one embodiment, the step of extracting features from each of the sEMG signals and the physiological signals may comprise a root-mean-square (RMS) feature extraction and a wavelength (WL) feature extraction. In another embodiment, the step of performing feature alignment includes synchronizing the sEMG signals and the physiological signals by using cross-correlation functions. In an additional embodiment, the step of correlating patterns recognized with pain intensity levels and classifying the pain intensity levels are performed using an artificial neural network classifier.

One of the unique and inventive technical features of the present invention includes the wearable mask for facial expression capture and for pain assessment, pain management, and clinical monitoring. Without wishing to limit the invention to any theory or mechanism, it is believed that the technical feature of the present invention advantageously provides for aligning embedded sensors on the mask with facial muscles that are activated when experiencing pain, thereby maximizing the signals detected by the sensors, and further enhancing the sensitivity of the system for measuring pain experienced by the patient.

Another unique and inventive technical feature of the present invention includes analyzing a plurality of physiological signals and comparing the signals with one another and/or a database to correlate the measured physiological signals with pain intensity values. In this way, an accurate measure of the pain levels experienced by the patients may be determined. By continuously monitoring the pain levels and displaying the detected pain levels, a medical provider may be able to make intelligent and effective pain management decisions for the patient, thereby improving quality of life in patients suffering from constant or complex pain, for example.

In addition, the current belief in the prior arts is that the incorporation of the sensors into a mask would interfere with detection. Although the sensors were localized, it was thought that the mask would couple the sensors together such that movement of one sensor would affect other sensors, resulting in noise and inaccurate signal detection. It was also thought that the mask would add significant weight to dislocate the sensors from the desired positions on the human face. Thus the prior art teaches away from the present invention. However, contrary to prior teachings, the embedding of the sensors into the mask of the present invention surprisingly worked and was able to detect signals related to pain expression from the individual facial muscles without exhibiting signal or placement issues. Furthermore, the multimodality resulting from the integration of surface electromyogram (sEMG) signals obtained by the wearable sensor mask and other physiological signals obtained by other sensor devices produced a synergistic effect that enhanced detection of the pain responses and distinguished them from other biological responses in the human. As such, none of the known prior references or work has the unique inventive technical features of the present invention.

Any feature or combination of features described herein are included within the scope of the present invention provided that the features included in any such combination are not mutually inconsistent as will be apparent from the context, this specification, and the knowledge of one of ordinary skill in the art. Additional advantages and aspects of the present invention are apparent in the following detailed description and claims.

### BRIEF DESCRIPTION OF THE DRAWINGS

The features and advantages of the present invention will become apparent from a consideration of the following detailed description presented in connection with the accompanying drawings in which:

FIG. 1A shows a wearable facial expression capturing system placed on a subject's face, where the system comprises a facial mask embedded with a plurality of electrodes, according to an embodiment of the present invention.

FIG. 1B is a non-limiting example of a prototype of the facial mask embedded with a plurality of electrodes.

FIG. 1C shows the prototype placed on a subject's face.

FIG. 1D shows another non-limiting example of wearable facial expression capturing system having a face mask with two electrodes.

FIG. 1E is another non-limiting example of wearable facial expression capturing system having a face mask with five electrodes.

FIG. 2 shows a non-limiting example a pain assessment system that continuously monitors pain from subjects and reports it to data fusion system which displays the data to a medical care provider.

FIG. 3 shows a non-limiting example of a pain assessment system that continuously monitors pain from subjects and reports it to a cloud and a remote database, which is then accessed by the medical care provider.

FIG. 4 shows a high-level flow chart depicting an example method for detecting biosignals from the plurality of electrodes and physiological signals and analyzing the signals to recognize facial expressions and deduce pain levels based on the facial expressions.

FIG. 5 shows an example plot showing surface electromyogram (sEMG) signals from eight channels of the system.

FIG. 6 shows an example scatter plot of the RMS features of four expressions from one fold training dataset with three of the four channels of sEMG signals.

FIG. 7 shows a schematic diagram of a pain stimulation and biosignal measurement environment.

FIG. 8 shows a timeline of a test for measuring pain intensity levels.

FIG. 9 shows a schematic diagram of a data processing flow and classification of matrices.

FIG. 10 shows Pearson's linear correlation coefficient between pain intensity levels and parameters in the matrices. The physiological parameters on the horizontal axis are sorted in descending order of coefficient of absolute value.

FIGS. 11A and 11B show distribution of area under curve (AUC) from classification with different number of sEMG parameters in addition to heart rate, breathe rate, and galvanic skin response.

### DESCRIPTION OF PREFERRED EMBODIMENTS

Following is a list of elements corresponding to a particular element referred to herein:
100 wearable facial expression capturing system
102 mask
104 electrode/sensor
106 connecting lead
108 sensor node
112 wireless transmitter
114 subject's face
115 eye recess or opening
116 forehead portion of mask
117 cheek portion of mask
118 lateral flap
200 pain assessment system
202 pain detection system
204 monitoring system
206, 212 patient
208, 214 wearable facial expression capturing system
210, 216 wireless transmitter
218 gateway
220 display
222 medical care provider
226 cloud
228 remote server
300 pain monitoring system
302 wearable facial expression capturing system
304 electrode/sensor
306 sensor node
308 wireless transmitter
310 processing module
312 wearable sensors
314 ECG sensor
316 breath rate sensor
318 heart rate sensor
320 PPG sensor
322 data fusion system
324 WIFI receiver
326 memory
328 processor
330 display

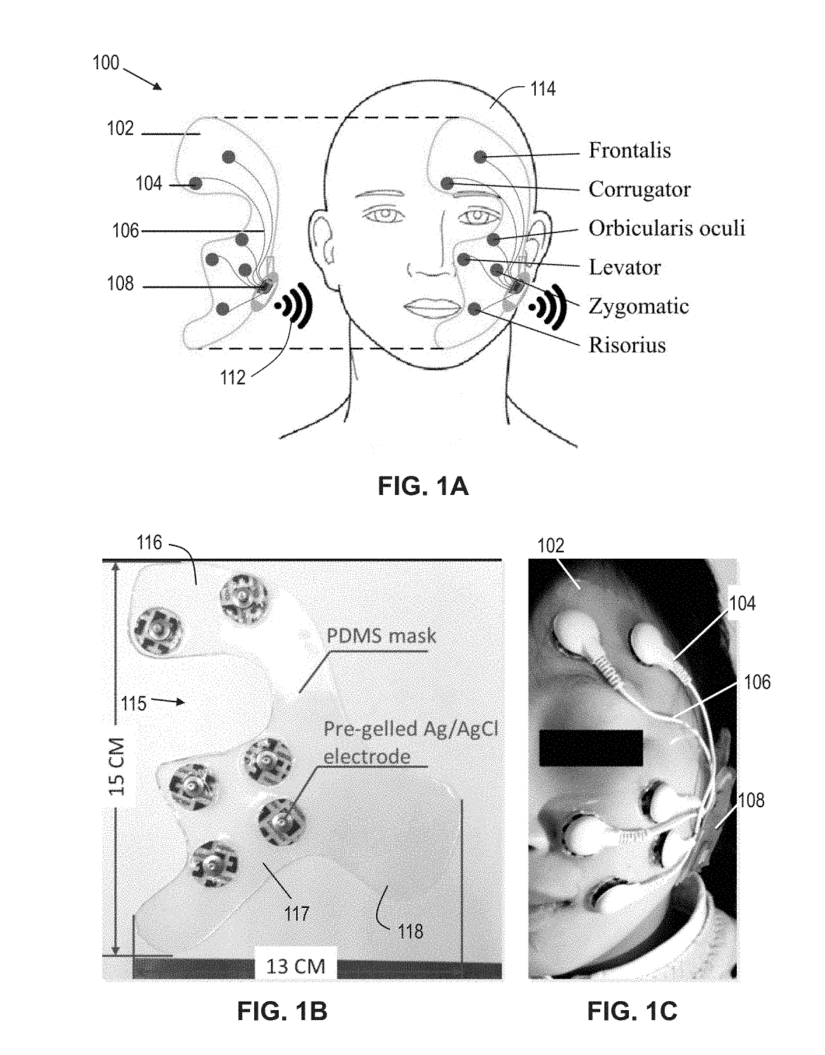

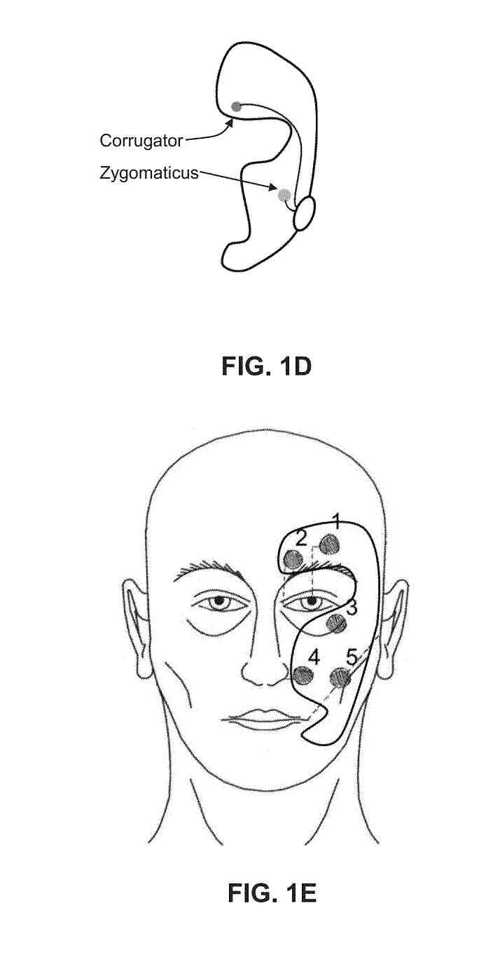

Referring now to FIG. 1A-11B, the present invention features a real-time pain monitoring system for subjects, who are unable to self-report, for example, to improve efficiency of reporting and optimizing pain management and medication. The present invention discloses a wearable facial expression capturing system (100) positioned over a subject, as shown in FIGS. 1A-1E. In some embodiments, the system (100) comprises a mask (102) made of a soft and pliable material, which can conform to the shape of the subject's face (114). As a non-limiting example, the mask (102) may be composed of poly dimethyl silicone elastomer (PDMS) substrate which is soft, stretchable, transparent, and light weight, and which can be worn on the face. As such, the softness of PDMS makes the mask fit well on the curvature of the user's face. Other materials may be used for creating the mask without deviating from the scope of the invention.

In some embodiments, a thickness of the mask (102) may be selected based on one or more of desired flexibility and overall weight for user comfort, for example. As a non-limiting example, the thickness of the mask (102) may be about 50-150 μm. In one non-limiting example, the thickness of the manufactured mask may be about 100 μm. As a non-limiting example, the overall weight of the mask may be about 7-10 g. In one non-limiting example, the weight of the mask may be about 7.81 g. Other values of thickness and weight may be used without deviating from the scope of the invention.

In some embodiments, the mask (102) is implemented by integrating detecting electrodes into the soft polydimethylsiloxane (PDMS) substrate. As a result, the designed mask is easy-to-apply, and offers a one-step solution, which can largely save the valuable time of the care givers when making setting up for sensing vital bio-signals from patients, in particular in the ICU ward environment. In a non-limiting embodiment, the mask (102) is integrated with a plurality of sensors or electrodes (104) embedded into the mask (102), such that when worn, the plurality of electrodes (104) are in contact with specific detection points on the subject's face (114). In one non-limiting example, the plurality of sensors (104) may include electrodes for detecting surface electromyogram (sEMG) signals from facial muscles. As an example, the electrodes may include six pre-gelled Ag/AgCl electrodes positioned at specific locations (positions 1-6 shown in FIG. 1A-1C) on the mask. In terms of pain related facial expressions, the main facial muscles that are involved are listed in Table I.

TABLE 1
Pain related facial muscles and the targeted facial action units (AU).

| Channel | Muscular basis | AU |
|---|---|---|
| 1 | Frontalis | |
| 2 | Corrugator | Brow lower (AU 4) |
| 3 | Orbicularis oculi | Lids tighten (AU 6) |
| | | Cheek raise (AU 7) |
| 4 | Levator | Nose wrinkle (AU 9) |
| | | Upper lip raiser (AU 10) |
| | | Eyes close (AU 43) |
| 5 | Zygomatic | Lip corner pull (AU 12) |
| 6 | Risorius | Horizontal mouth stretch (AU 20) |

In some embodiments, fewer electrodes may be used to detect biosignals from the facial muscles to recognize facial expressions. As a non-limiting example, four electrodes may be positioned to line up with the corrugator, orbicularis oculi, levator, and the zygomatic to study the facial expressions. In other embodiments, as shown in FIG. 1D, the facial mask may include two sensors positioned to line up with the corrugator and the zygomatic. In another non-limiting example, as shown in FIG. 1C, the facial mask may include five sensors positioned to line up with corrugator, frontalis, orbicularis oculi, levator, and the zygomatic. In alternative embodiments, additional reference electrodes may be included in the mask. The reference electrode may be positioned on the bony area behind the ear, for example.

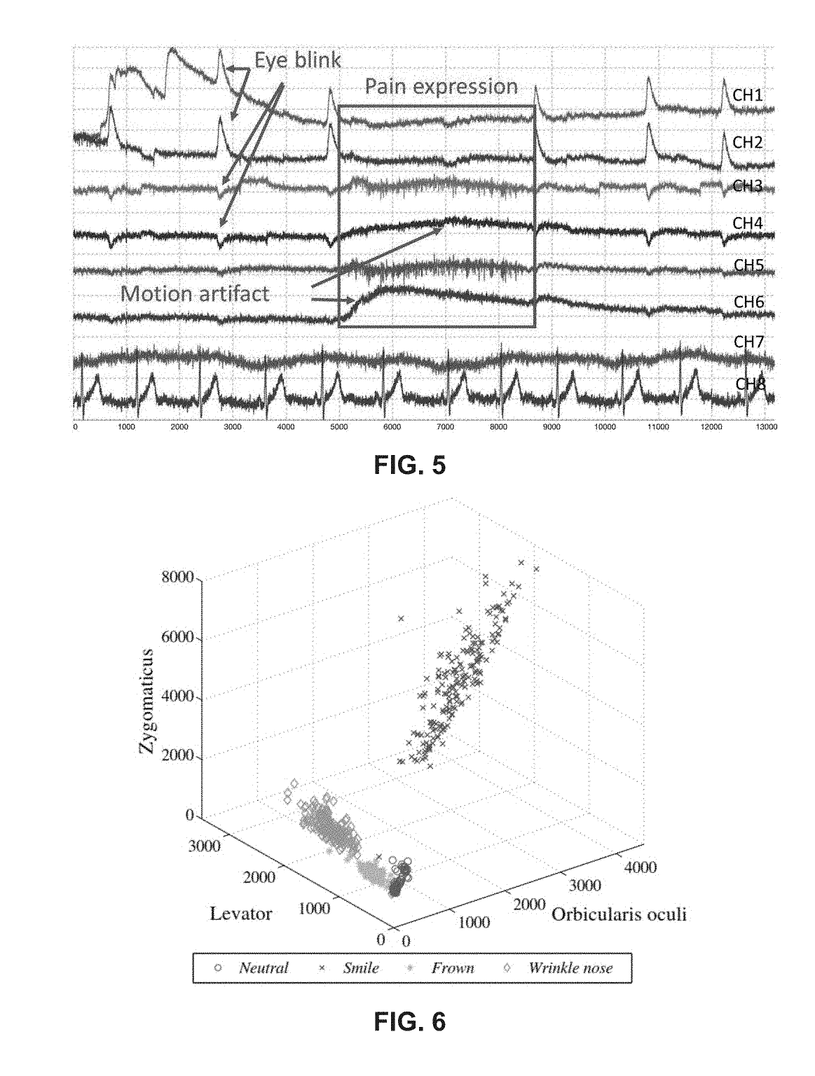

To recognize facial expressions with sEMG method, three to eight channels of sEMG signals may be used. An example plot showing sEMG signals from eight channels is shown in FIG. 5. The sEMG signals may be analyzed to ascertain the facial expression, as described further below.

Each electrode (104) is aligned with the facial muscles of table 1. Herein, a spacing between individual electrodes is selected such that each electrode overlays on top of a muscle from table 1. Each electrode (104) is integrated on the inner side surface of the mask (102) and closely attached to facial skin for reliable surface electromyogram (sEMG) measurement. The placement of the electrodes is determined by the targeted facial muscles. Due to the soft nature of the implemented mask, the electrode position and the shape of the mask can be slightly adjusted accordingly to accommodate individual facial differences.

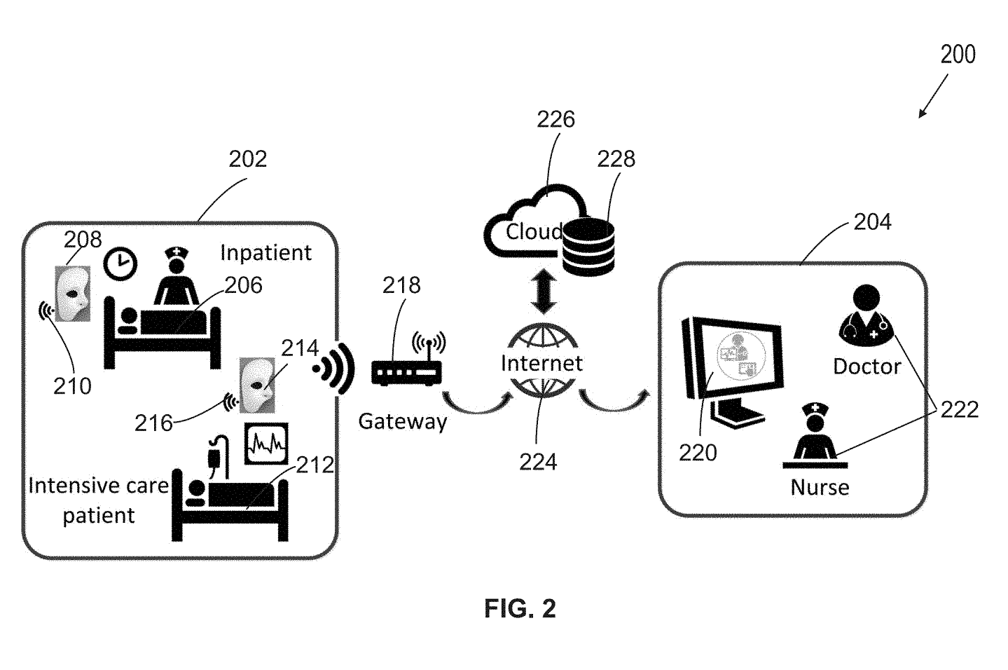

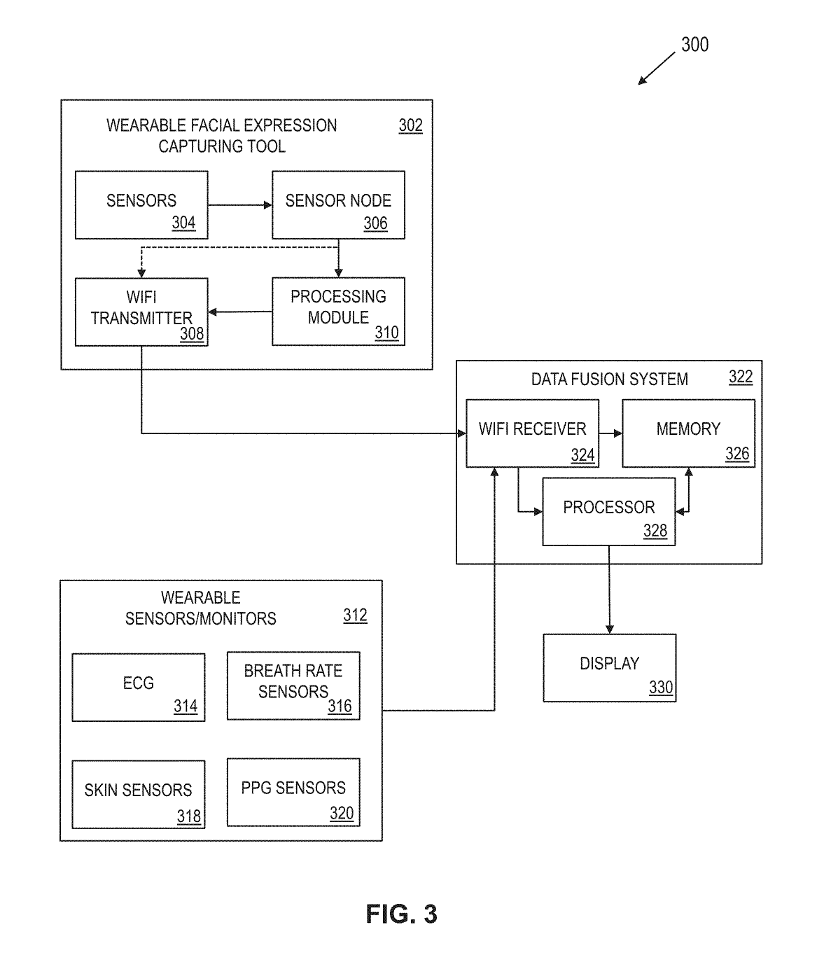

Each electrode (104) is electrically coupled to a sensor node (108) via connecting leads (106). As an example, the connecting leads (106) may be snapped or clipped on to the electrode (104) embedded on the mask (102). Herein, the connecting leads may be positioned along a top surface of the mask (102). The sensor node (106) may receive biosignals or sEMG signals detected by the electrodes via the connecting leads (106). The sensor node (108) may include a processing module that is configured for conditioning and digitizing the biosignals. The sensor node (108) may additionally include a wireless transmitter (112) that is configured to wirelessly transmit the biosignals to a receiver end, as shown in FIGS. 2 and 3. In one non-limiting example, the sensor node (108) may be attached behind the ear. In other examples, the sensor node (108) may be positioned on the neck. As such, the sensor node (106) may be positioned at other locations without deviating from the scope of the invention.

Turning now to FIG. 2, a schematic diagram of an example pain assessment system (200) that continuously monitors pain from several subjects and reports the data to a medical care provider (222) for pain management is shown. The system (200) comprises of a signal detection system (202) which detects biosignals from multiple patients each wearing a wearable facial expression capturing system (208, 214). The wearable facial expression capturing systems (208, 214) may be non-limiting examples of the wearable facial expression capturing system (100) shown in FIG. 1. For example, the detection system (202) may detect biosignals from a first patient (206) wearing the wearable facial expression capturing system (208) and may transmit the biosignals of the first patient (206) wireless through a wireless transmitter (210) of the system (208) to a cloud (226) or remote server (228) wirelessly via a gateway (218). The detection system (202) may additionally detect biosignals from a second patient (212) wearing the wearable facial expression capturing system (214) and may transmit the biosignals of the second patient (212) to the cloud (226) or remote server (228) wirelessly via the gateway (218). In cloud (226) or server (228), signals from the signal detection system (202) may be processed and classified after which it is sent to a monitoring system (204) where the signals are displayed to a medical care personnel (220) such as a nurse or doctor via a display (220). Herein, the display may include any device that is capable of visually displaying the signals such as a monitor, mobile phone, laptop, table, for example. Processing of the biosignals may include filtering, segmenting, and performing feature extraction, as described below.

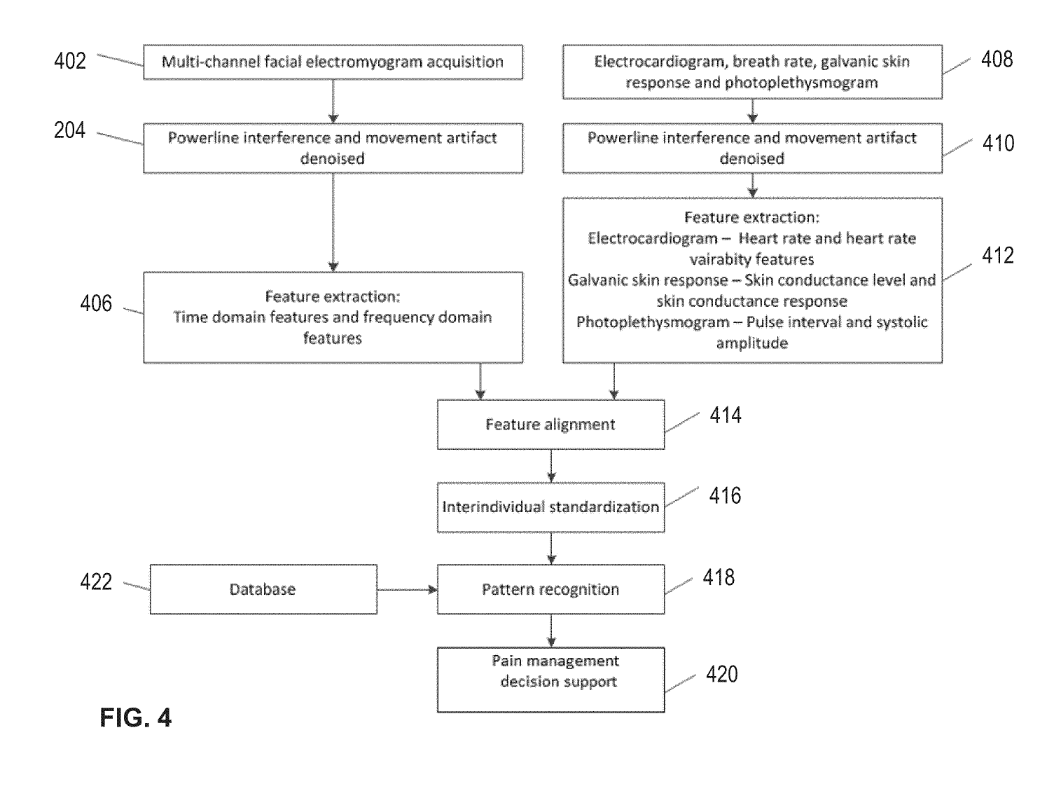

Current acute pain intensity assessment tools are mainly based on self-reporting by patients, which is impractical for non-communicative, sedated or critically ill patients. The present invention discloses a continuous pain monitoring systems and methods with the classification of multiple physiological parameters, as shown in FIGS. 3 and 4. Turning now to FIG. 3, a schematic diagram of a pain monitoring system (300) is shown. The pain monitoring system (300) may include a wearable facial expression capturing system (302), additional wearable sensors (312) and a data fusion system (322). The wearable facial expression capturing system (302) may be a non-limiting example of the wearable facial expression capturing system (100) described in FIG. 1. As described previously, the wearable facial expression capturing system (302) may include a mask that is placed on a subject's face. The mask may include a plurality of embedded sensors (304), a sensor node (306), a processing module (310), and a WIFI transmitter (308). As explained previously, the system (302) may detect biosignals or biopotentials or sEMG from the surface of the face. Herein, the sEMG signal is a voltage produced by the facial muscles, particularly muscle tissue during a contraction.

In some embodiments, facial sEMG signals may be gathered when the person is with a neutral expression and facial expressions such, smile, frown, wrinkle nose, and the like. The sEMG signals detected by the plurality of sensors (304) may be sampled as different channels. As an example, when four electrodes are placed on the muscles to detect sEMG signals, four channels may be sampled at 1000 SPS. After the sampling, the signals may be filtered. In a non-limiting example, the sampled signals may be filtered using a 20 Hz high pass Butterworth filter and a 50 Hz notch Butterworth filter. As such, the filtering of the signals reduces the artifacts and power line interference coupled to the connecting leads. The sEMG signals may be segmented into 200 ms slices, for example. In some embodiments, the sEMG signals may be filtered by the processing module (310). In some embodiments, the sEMG signals may be transmitted to a removed server/cloud, as shown in FIG. 3, where the signals are analyzed. In some embodiments, the sEMG signals may be transmitted to a data fusion system (322) for further analysis. The data fusion system (322) may include a WIFI receiver (324) configured to receive the sEMG signals from one or more systems (302) and the remote server/cloud. The data fusion system (322) may additionally include a memory (326) and a processor (328) for storing and performing processing steps, as disclosed below.

In some embodiments, the data fusion system (322) may receive raw sEMG signals from the system (302), and the processor (328) may filter and segment the sEMG signals. In some embodiments, the data fusion system (322) may receive filtered and segmented sEMG signals.

Once the sEMG signals and filtered and segmented, a root-mean-square (RMS) feature extraction may be performed on the signals. Mathematically, the RMS features are extracted using the following equation:

$$RMS = \sqrt{\frac{1}{N} {\sum }_{i = 1}^{N} {x}_{i}^{2}} \qquad ( 1 )$$

The RMS feature extraction may provide insight on sEMG amplitude in order to provide a measure of signal power, for example. Wavelength (WL) feature extraction may be additionally or alternatively performed on the sEMG signals as a measure of signal complexity. WL features are extracted using the following equation:

WL=Σᵢ₌₁^{N-1}|xᵢ₊₁−xᵢ| (2)

A multivariate classifier is trained for expression classification. Parameters of Gaussian distribution for each expression are estimated from training data, i.e. a feature matrix. Herein, the feature matrix may include signals for neutral, smile, frown, wrinkle nose, and the like. In some embodiments, the feature matrix may be stored in the memory (326) of the data fusion system (322).

Then the posterior probability of a given class c in the test data is calculated for pattern recognition. The equation below is Bayes theorem for the univariate Gaussian, where the probability density function of continuous random variable x given class c is represented as a Gaussian with mean μ_{c} and variance σ_{c}2.

$$P ( c \vert x ) ∝ \frac{1}{2 {πσ}_{c}} exp ( \frac{- {( x - {μ}_{c} )}^{2}}{2 {σ}_{c}^{2}} ) P ( c ) \qquad ( 3 )$$

In this way, the sEMG signals may be compared with the feature matrix, and the facial expression may be recognized based on the comparison. As an example, when employing multivariate Gaussian classifier, 10 fold cross-validation is applied and the classification accuracy is about 82.4%. The scatter plot of the RMS features of four expressions from one fold training dataset with three of the four channels sEMG are shown in FIG. 6. Each test dataset is combined by four expressions in the sequence of neutral, smile, frown and wrinkle nose.

In addition to extracting facial expressions based on sEMG signals detected from the facial expression capturing system (302), the pain monitoring system (300) may be configured to receive signals from other wearable sensors/monitors (312). Some non-limiting example of the wearable sensors/monitors include heart rate (HR) sensors, breath rate (BR) sensors, galvanic skin sensors, photoplethymogram (PPG) sensors, and the like. As an example, the wearable sensor (312) may be a watch that is worn on the wrist and monitors the heart rate. As another example, the wearable sensor (312) may be a monitor that is worn on a chest and torso for monitoring the heart rate. As yet another example, the wearable sensor may be the PPG sensor worn on a finger to monitor pulse oxygen in the blood. Other examples of wearable sensor include biopatches and electrodes worn/attached to anywhere on the body.

The pain monitoring system may receive signals from the wearable sensor (312). Herein, the signals received may include one or more of a heart rate (HR), a breath rate (BR), a galvanic skin response (GSR), a PPG signal, and the like. The processor (328) may filter the signals received from one or more of the wearable sensors (312) to remove powerline interference and remove movement artifacts. The processor (312) may additionally perform feature extraction on the signals received from the wearable sensors. Some examples of the feature extraction may include extracting heart rate and heart rate variability features from the ECG, extracting skin conductance level and skin conductance response from the skin sensors, and extracting pulse interval and systolic amplitude from the PPG signal. Other features may be extracted without deviating from the scope of the invention. The processor may combine the sEMG feature extraction and the sensor feature extraction to monitor and manage pain, as described in FIG. 4.

Turning to FIG. 4, an example method (400) for integrating sEMG signals with additional biosignals to monitor pain levels in subjects is shown. Instructions for carrying out method 400 included herein may be executed by a processor based on instructions stored on a memory of the processor and in conjunction with signals received from sensors of the pain monitoring system, such as the sensors described above with reference to FIGS. 1A-3. Method 400 includes acquiring multi-channel facial sEMG signals at 402. As described previously, the sEMG signals may be detected using a wearable facial expression capturing system such as system (100) described in FIGS. 1A-1E. At 404, method 400 includes powerline interference and movement artifact denoising and method (400) proceeds to 406, where the facial features are extracted, as described in detail with reference to FIG. 3. As an example, the denoising may include removing powerline interference and movement artifact from the signals. For most of raw biopotential data, contamination by the environment noise or human body movement is inevitable. One common contamination source among biopotential signals is power line interference, composed with 50 Hz or 60 Hz and its harmonics. Another common noise source in EMG is body movement that dominates low frequency part of the signal. Therefore, denoising is the basic processing applied in biopotential signals. A variety of filters from FIR or IIR, adaptive ones, to wavelet method can be applied in terms of noise cancellation in order to improve signal to noise rate.

At 406, the feature extraction may include extracting time domain and frequency domain features of the sEMG signals using RMS and WL features, as described in equations (1) and (2).

Method 400 may simultaneously receive and process physiological signals from other wearable devices as described with reference to FIG. 3. For example, at 408, method 400 includes receiving one or more of HR, BR, GSR, and PPG signals from wearable devices (such as devices (312 shown in FIG. 3). Like step 404, at 410, method 400 includes denoising the signals received at 408. Next, at 412, method 400 includes extracting features from the signals of the wearable sensors. Some examples of the feature extraction may include extracting heart rate and heart rate variability features from the ECG, extracting skin conductance level and skin conductance response from the skin sensors, and extracting pulse interval and systolic amplitude from the PPG signal. In some embodiments, RMS and/or WL feature extraction (equations (1) and (2)) may be performed on the signals from the wearable sensors to extract the features.

At 414, method 400 includes performing time alignment on the features extracted from sEMG signals, and from the signals such as HR, BR, GSR, PPG, and the like. As such, the sEMG, HR, BT, GSR, and PPG measurements may include signals collected asynchronously by multiple sensors. In order to integrate the signals and study them in tandem, the signals have to be synchronized. In one non-limiting example, the sEMG signals may be aligned with the HR, BR, GSR, PPG signals using cross-correlation functions. Other techniques may be used to synchronize the signals, without deviating from the scope of the invention.

At 416, method 400 includes performing interindividual standardization or normalization. The interindividual standardization includes rescaling the range and distribution of each signal. Rescaling may be used to standardize the range of the sEMG signals and the physiological signals. As such, the standardization of the signals may reduce subject-to-subject and trial to trial variability. In one embodiment, the signals may be standardized by equation (4) shown below:

$$Z = \frac{( X - μ )}{σ} \qquad ( 4 )$$

where X is the feature, p is the mean, and a is the standard deviation. The standardization results in generating a parameter matrix. As an example, the standardization of the sEMG signals may result in a matrix containing one set of RMS features and another set of WL features. For example, for sEMG signals arising from five face muscles, the parameter matrix may include ten standardized values. In addition, the parameter matrix includes standardized physiological signals such as HR, BR, and GSR. Thus, the standardization of the sEMG signals and the physiological signals may generate a 13-dimensional parametric matrix.

At 418, method 400 includes performing pattern recognition. The sEMG signals and the BR, HR, and GSR, signals may be compared with corresponding feature matrices stored in the database (422). Based on the comparison, method 400 may classify the signals into no pain, mild pain, or moderate/severe pain. Herein, the parameters of a built model may be trained by the existing database. The model may then be used to classify the new coming features. The model may also be later updated by retraining with the updated database which involves the labelled new coming features. In one embodiment, the comparison may include performing correlation analysis between the physiological parameters, sEMG, and pain intensity levels. As an example, GSR, HR, and BR in the parameter matrix may be used as predicting. Herein, GSR and HR positively correlated with pain intensity level, indicating that these two parameters were more likely to increase when a healthy subject experiences a high intensity of pain, while BR decreases. Among facial sEMG parameters, ZygRMS includes greater correlation to the pain intensity level than others. GSR, HR, BR and two corrugator superclii parameters in the median matrix showed stronger correlation to the pain intensity level than the parameter matrix. As such, the medians of both corrugator supercilii parameters showed considerable potential for differentiating pain intensity levels. Thus, transient response of facial expressions may correlate to acute pain. In some embodiments, Pearson's linear correlation analysis may be used to compare the sEMG signals and physiological signals with pain intensity levels.

Thus, the present invention discloses automatic pain monitoring by classification of multiple physiological parameters. In addition, by performing parameter matrix classification where the physiological parameter samples are classified every second, it may be possible to continuously monitor pain. The physiological parameters are either clinically accessible or available from wearable devices and are appropriate for continuous and long term monitoring. Besides, this monitoring method may help clinicians and personnel working with patients unable to communicate verbally to detect their acute pain and hence treat it more efficiently.

Examples of Medical Use Cases: Post-Operative Pain Assessment and Patient Behavior Assessment (e.g., Blink and Swallowing)

The automatic pain detecting system and method disclosed herein may be used to detect pain in non-communicative subjects. As an example, in emergency rooms or in ambulances, where patients are sometimes unable to communicate, the present invention may be used to automatically detect the level of pain that the patient is experiencing. As another example, for premature babies or infants or people with cognitive disabilities such as Alzheimer's or dementia, the present invention may be used to automatically detect the level of pain experienced by the subject. Once the pain levels are determined, the medical care provider may be able to administer the proper treatment, or prescribe the correct levels of pain medications, for example.

In some situations, the medical provider may need to assess if the pain is real. For example, in subjects who are opioid/substance users, the medical provider cannot rely on the communication from the subjects. There needs to be an independent and more accurate measure of pain levels, so that the medical provider may be able to corroborate the results with the verbal communication received from the subjects. In this way, the medical provider may be able to selectively prescribe pain medications only when the pain is real.

The present invention may be used in situations to regulate the pain medication dosage. As an example, in postoperative patients who need persistent pain prevention, the present invention may be used to automatically detect the pain levels, thereby providing the medical care provider with an accurate measure of the pain levels experienced by the patients, so that the provider can adjust the dosage of the pain medications based on the measured pain levels. In some examples, the present invention may be used to assess pain in palliative or home care patients. In some more examples, the present invention may be used for detection/prevention of breakthrough pain in cancer. The present invention may also be used to detect work related stress and other unhealthy distress experienced by subjects.

### Example

The following is a non-limiting example of the present invention. It is to be understood that said example is not intended to limit the present invention in any way. Equivalents or substitutes are within the scope of the present invention.

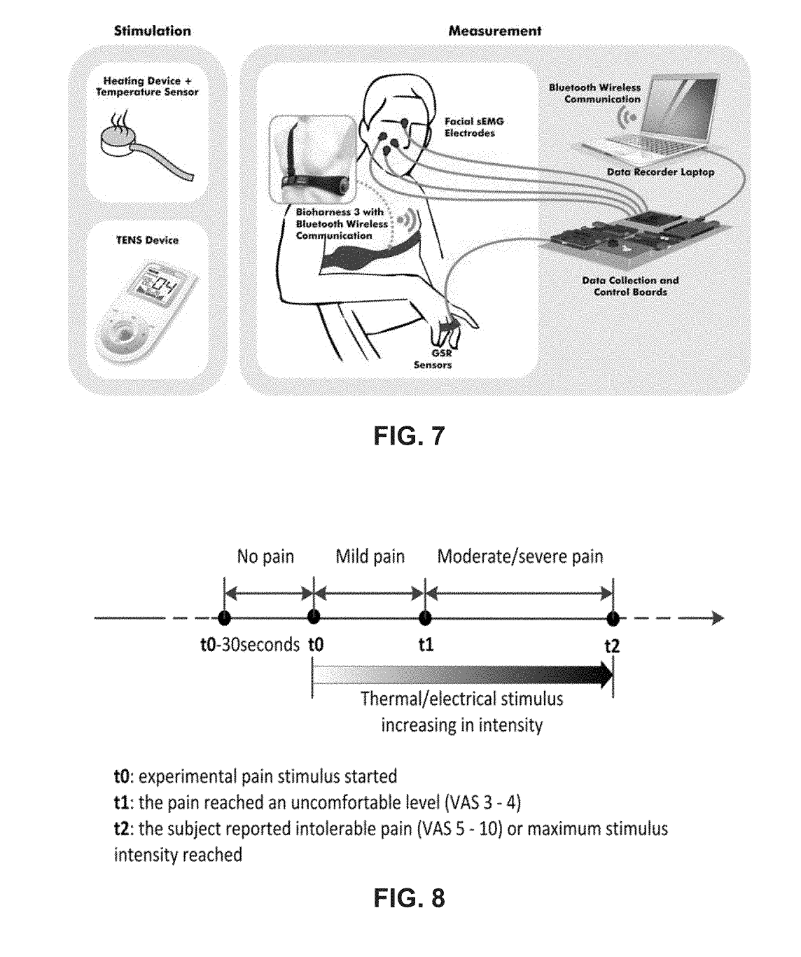

To develop a continuous pain monitoring method from multiple physiological parameters with machine learning, HR, BR, GSR and facial surface electromyogram (sEMG) were monitored from healthy volunteers under experimental pain stimulus (FIG. 7). Facial expressions were captured from sEMG of the skin above five pain expression-related facial muscles: corrugator supercilii, orbicularis oculi, levator labii superiors, zygomaticus major and risorius. Two types of experimental pain stimuli, thermal stimuli (heat) and electrical stimuli, were employed on both the right and left sides of the body in the study to cover more than one dimension of pain perceptions. Three pain intensity levels—no pain, mild pain, and moderate/severe pain—were collected from self-reports with visual analogue scale (VAS) and were defined as three categories in classification (shown in FIG. 8).

### Biopotential Measurement

Physiological signals including HR, BR, GSR and five facial sEMG from the right side of the face were continuously recorded throughout the session. FIG. 7 shows a brief description of the measurement environment, where GSR was captured from pre-gelled Ag/AgCl electrodes on the finger, five channels sEMG were captured from the Ag/AgCl electrodes on corrugator supercilii, orbicularis oculi, levator labii superiors, zygomaticus major and risorius on the face, and HR and BR were from a Bioharness® belt worn on chest. HR, BR and GSR were taken at one second time resolution and sEMG were sampled with a Texas Instruments 8 channel biopotential measurement device at a rate of 1000 samples per second.

### Study Design

The study subject was seated in an armchair. At the beginning of the study session, the sensors and the device were established and it was ensured that they were able to record and appropriately catch the signals from all devices. The pain was induced by thermal and electrical stimuli in a random fashion, two times for each stimuli. The subjects were tested four times during each session and the tests were 1) electrical stimuli on the right-hand ring finger, 2) electrical stimuli on the left-hand ring finger, 3) thermal stimuli on the right inner forearm, and 4) thermal stimuli on the left inner forearm. The pain exposure starting location was randomized and the change of stimulated skin site helped in avoiding habituation to repeated experimental pain. Each data collection session started by letting the subject settle down and rest for ten minutes, so as to acquaint himself or herself with the study environment. Pain testing was only repeated after the subject's HR and BR had returned (if changed) to their respective baseline level.

The intensity of pain was evaluated using VAS at two time points: t1—when the pain reached an uncomfortable level (VAS 3-4), and t2—when the study subject reported intolerable pain or when stimulus intensity reached the non-harmful maximum. The time points and data definition are illustrated in FIG. 8. To balance the data size of each class, data of the 30 seconds before applying pain stimulus was labelled as no pain. During pain stimulation, data from when it started to when it reached an uncomfortable level was labelled as mild pain. The second part of the data under pain stimulus was marked as moderate/severe pain, where either moderate or severe depends on the VAS the study subject reported. All physiological signals were marked with time stamps and were saved for offline processing along with VAS evaluations.

### Data Pre-Processing

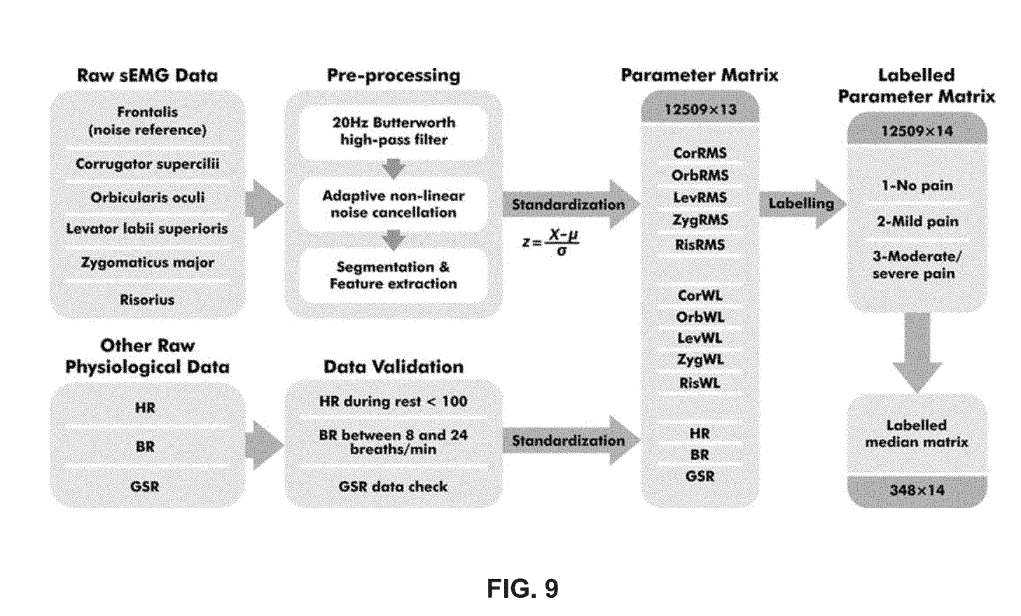

Data on sEMG and other physiological data were processed and checked separately, as shown in FIG. 9. The aim of the pre-processing was to eliminate noise interference and verify the validation of the data. For sEMG, 50 Hz power line noise was coupled to electrode lead wires from the environment. Movement artifacts and baseline drift in low frequencies both caused noise in the sEMG signal. There was also a third noise source, which was caused by electrical stimulus pulses. Electrical pulses were added to finger skin's surface and captured from facial skin's surface as well, due to the electrical conductivity of the human body. In sEMG pre-processing, a 20 Hz Butterworth high-pass filter was first applied to remove movement artifacts and baseline drift from six sEMG channels. Adaptive noise cancellation was employed for the power line and electrical pulse elimination, where non-linear noise in each of the five pain-related facial muscle channels was estimated by reference to a frontalis sEMG with an adaptive neuro-fuzzy inference system (ANFIS) estimator.

To unify the time granularity of sEMG data and other physiological data, sEMG data was split into 1000-sample segments for feature extraction. The root mean square (RMS) in equation (1) and wavelength (WL) in equation (2) were the chosen features, where N was the window length and xi was the i^{th} data point in the window. The RMS feature provided direct insight on sEMG amplitude in order to provide a measure of signal power, while WL was related to both waveform amplitude and frequency [30]. All signal processing was conducted in MATLAB.

For all physiological features, data validation on range and constraint were carried out. After checking, three thermal stimuli tests were excluded from the total of 120 tests due to invalid GSR data in the no pain part and another thermal stimulus test was excluded for invalid sEMG data. All the validated physiological features were standardized with a standard core within each test and constituted the 13-dimensional parameter matrix. This standardization rescaled the range and distribution of each parameter, in which way the within-subject and between-subject difference in value range was suppressed. There were 12,509 samples at one second resolution from 116 tests in the parameter matrix. Each sample with 13 parameters was labelled according to the data division in FIG. 2. No pain, mild pain and moderate/severe pain data were labelled as 1, 2 and 3 respectively. Subsequently, the statistical median of every parameter was calculated from three sections of each test and constituted the median matrix with a length of 348.

### Data Observation and Classification

To visualize the median matrix in 2-dimensional scatter plots, the dimension of parameters in the median matrix was first reduced from 13 with principal component analysis. The first two principal components of the median matrix were non-normally distributed. Nevertheless, with the ability of multivariate analysis, Gaussian distributions were then estimated for each pain intensity level to observe their approximate distribution boundaries in the first two principal components. To fit Gaussians to the parameters of each group, the mean (μ) and variance (σ2) of Gaussian distribution were estimated in maximum likelihood estimation. In a d-dimensional Gaussian distribution, mean and variance were estimated from

$$\begin{matrix}{\hat{μ}}_{i} = \frac{1}{N} {\sum }_{n = 1}^{N} {x}_{ni} , for i = 1 , … d & ( 5 ) \\ {\hat{σ}}_{ij} = \frac{1}{N} {\sum }_{n = 1}^{N} ( {x}_{ni} - {\hat{μ}}_{i} ) ( {x}_{nj} - {\hat{μ}}_{j} ) , for i = i , j = 1 , … d & ( 6 )\end{matrix}$$

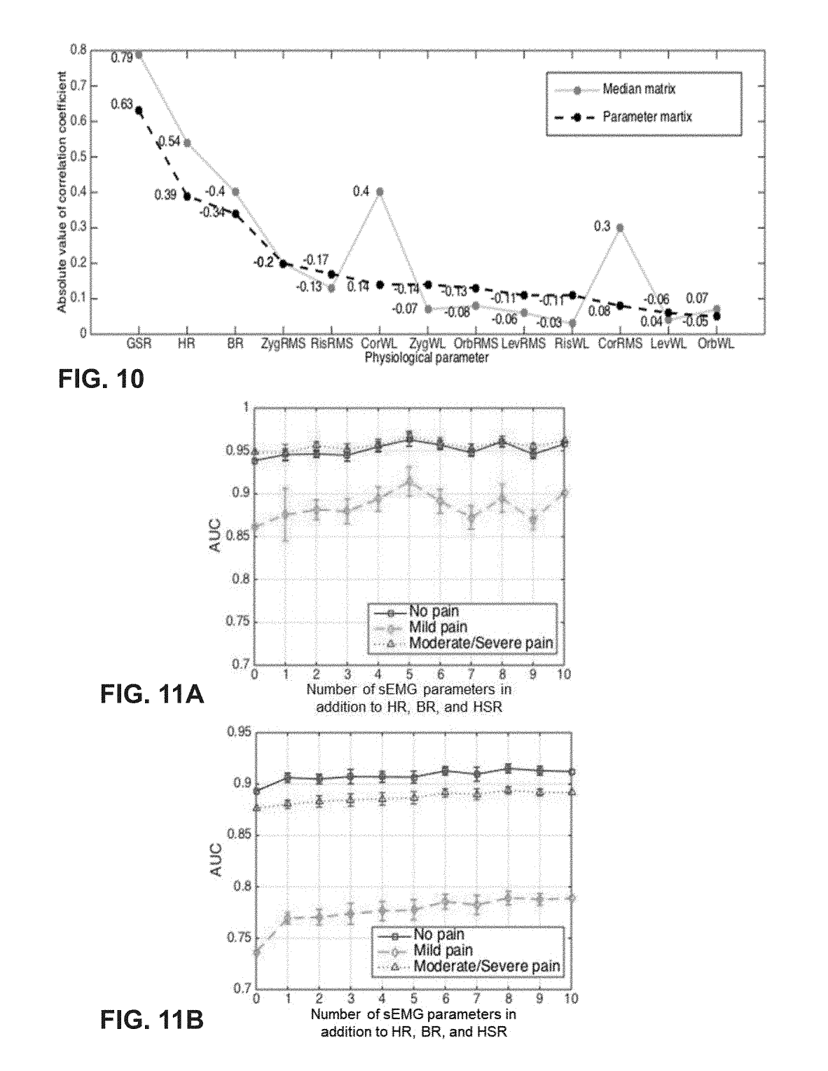

The 95% confidence regions of distributions were marked as approximate boundaries. Tests with different pain stimuli were plotted separately. The significance of each parameter in pain intensity level recognition was observed with correlation analysis. Pearson's linear correlation coefficients between each standardized parameter and labels were calculated, as shown in FIG. 10.

Using the classification method in machine learning, a model can be built to predict class labels (i.e. 1—No pain, 2—Mild pain and 3—Moderate/Severe pain) from input features (i.e. parameter matrix or median matrix). The resulting classifier is then used to assign class labels to the testing instance with new input features. One benefit of applying classification is its effectiveness in establishing many-to-many mapping. The classification technique chosen in this study was artificial neural network (ANN), which is a non-linear classifier having generally better performance with continuous and multi-dimensional features. This method emulates the information processing capabilities of human brain neurons and can provide flexible mapping between inputs and outputs.

With 13 parameters as the classifier inputs and 3 pain intensity levels as the outputs, the ANNs classifier was built in three layers: an input layer with 13 units, a hidden layer with 10 units and an output layer with 3 units. The classifier was applied to both the labelled median matrix and the labelled parameter matrix. Before classification, the samples were divided randomly into three proportions, where 70% were training samples being presented initially to the classifier for training the network; 15% were validation samples to improve classifier generalization properly; and the remaining 15% were testing samples, independent from the trained classifier for classifier performance measurement. The classifier in this work was trained and evaluated in MATLAB Neural Network Toolbox®. The receiver operating characteristic (ROC) curve of each classification was presented. Both average accuracy and the area under ROC curve (AUC) were evaluated as the performance of classification. The true positive rate (TPR) was also taken into consideration in the evaluation, indicating the correct recognition rate of each pain intensity level. The distributions of AUC in classification with different number of involved parameters are shown in FIGS. 11A and 11B.

Thus, patterns of self-reported acute pain intensity levels from monitored physiological signals were observed, which were categorized into no pain, mild pain and moderate/severe pain based on reported VAS.

As used herein, the term “about” refers to plus or minus 10% of the referenced number.

Various modifications of the invention, in addition to those described herein, will be apparent to those skilled in the art from the foregoing description. Such modifications are also intended to fall within the scope of the appended claims. Each reference cited in the present application is incorporated herein by reference in its entirety.

Although there has been shown and described the preferred embodiment of the present invention, it will be readily apparent to those skilled in the art that modifications may be made thereto which do not exceed the scope of the appended claims. Therefore, the scope of the invention is only to be limited by the following claims. In some embodiments, the figures presented in this patent application are drawn to scale, including the angles, ratios of dimensions, etc. In some embodiments, the figures are representative only and the claims are not limited by the dimensions of the figures. In some embodiments, descriptions of the inventions described herein using the phrase “comprising” includes embodiments that could be described as “consisting of”, and as such the written description requirement for claiming one or more embodiments of the present invention using the phrase “consisting of” is met.

The reference numbers recited in the below claims are solely for ease of examination of this patent application, and are exemplary, and are not intended in any way to limit the scope of the claims to the particular features having the corresponding reference numbers in the drawings.

What is claimed is:

1. A method for integrating surface electromyogram (sEMG) signals and physiological signals for automatically detecting pain intensity levels experienced by a human, wherein the method comprises:
(a) providing a wearable facial expression capturing system (100) for measuring said pain intensity levels, the system (100) comprising:
(i) a flexible mask (102) contoured to at least partially cover one side of the human's face (114), the mask having an eye recess or opening (115) disposed between an elongated forehead portion (116) of the mask, which is above the eye recess (115), and a cheek portion (117) of the mask, which is beneath the eye recess (115);
(ii) at least two sensors (104) disposed in the mask (102), wherein a first sensor is disposed in the forehead portion of the mask (116), and a second sensor is disposed in the cheek portion of the mask (117);
(iii) a sensor node (108) disposed on a lateral flap (118) extending from the cheek portion of the mask, wherein the sensor node (108) comprises a processing module and a transmitter (112); and
(iv) connecting leads (106) electrically coupling each of the at least two sensors (104) to the sensor node (108);
(b) applying the flexible mask to partially cover one side of the human's face such that the first sensor aligns with a corrugator facial muscle and the second sensor aligns with a zygomatic facial muscle;
(c) detecting sEMG signals from the corrugator facial muscle and the zygomatic facial muscle via the first and second sensors, respectively;
(d) filtering the detected sEMG signals via the processing module;
(e) transmitting the filtered sEMG signals to a data processing system (322) via the wireless transmitter (308);
(f) receiving physiological signals transmitted from one or more wearable sensors (312) to the data processing system (322), the physiological signals comprising one or more of a breath rate, a heart rate, a galvanic skin response (GSR), or a photoplethysmogram (PPG) signal;
(g) extracting features from each of the sEMG signals and the physiological signals;
(h) performing feature alignment on features extracted from the sEMG signals and the physiological signals;
(i) performing interindividual standardization on each of the sEMG signals and the physiological signals;
(j) performing pattern recognition by comparing the sEMG signals and the physiological signals to a database;
(k) correlating patterns recognized with pain intensity levels and classifying the pain intensity levels; and
(l) displaying the pain intensity levels to a medical care provider, thus allowing for continuous and automatic pain monitoring.

2. The method of claim 1, wherein extracting features from each of the sEMG signals and the physiological signals comprises a root-mean-square (RMS) feature extraction and a wavelength (WL) feature extraction.

3. The method of claim 1, wherein performing feature alignment includes synchronizing the sEMG signals and the physiological signals by using cross-correlation functions.

4. The method of claim 1, wherein an artificial neural network classifier correlates patterns recognized with pain intensity levels and classifies the pain intensity levels.

5. The method of claim 1, wherein the flexible mask (102) is composed of polydimethyl silicone elastomer (PDMS).

6. The method of claim 1, wherein the sensors (104) comprise Ag/AgCl electrodes.

7. The method of claim 6, wherein the electrodes are formed on an inner surface of the mask (102) such that the electrodes are directly contacting skin when the mask is applied to the human's face.

8. A method for integrating surface electromyogram (sEMG) signals and physiological signals for automatically detecting pain intensity levels experienced by a human, wherein the method comprises:
(a) receiving the sEMG signals from a wearable facial expression capturing system (302) placed on the human's face (114), wherein said system (302) comprises (i) a mask embedded with a plurality of sensors (304) at locations that line up with specific facial muscles, wherein the sensors are configured to detect sEMG signals from the facial muscles; (ii) a sensor node (306) configured to analyse the sEMG signals detected by the electrodes; (iii) a processing module (310) configured to filter the sEMG signals; and (iv) a wireless transmitter (308) configured to wirelessly transmit the filtered sEMG signals to a data processing system (322);
(b) receiving physiological signals transmitted from one or more wearable sensors (312) to the data processing system (322), the physiological signals comprising one or more of a breath rate, a heart rate, a galvanic skin response (GSR), or a photoplethysmogram (PPG) signal;
(c) extracting features from each of the sEMG signals and the physiological signals;
(d) performing feature alignment on features extracted from the sEMG signals and the physiological signals;
(e) performing interindividual standardization on each of the sEMG signals and the physiological signals;
(f) performing pattern recognition by comparing the sEMG signals and the physiological signals to a database;
(g) correlating patterns recognized with pain intensity levels and classifying the pain intensity levels; and
(h) displaying the pain intensity levels to a medical care provider, thus allowing for continuous and automatic pain monitoring.

9. The method of claim 8, wherein extracting features from each of the sEMG signals and the physiological signals comprises a root-mean-square (RMS) feature extraction and a wavelength (WL) feature extraction.

10. The method of claim 8, wherein performing feature alignment includes synchronizing the sEMG signals and the physiological signals by using cross-correlation functions.

11. The method of claim 8, wherein correlating patterns recognized with pain intensity levels and classifying the pain intensity levels is done by an artificial neural network classifier.

12. A facial expression capturing system (100) for measuring pain levels experienced by a human, the system (100) comprising:
a) a flexible mask (102) contoured to at least partially cover one side of the human's face (114), the mask having an eye recess or opening (115) disposed between an elongated forehead portion (116) of the mask, which is above the eye recess (115), and a cheek portion (117) of the mask, which is beneath the eye recess (115);
b) six sensor positions located on the mask (102) such that two sensor positions are located laterally on the elongated forehead portion (116) of the mask and the other four sensor positions located on the cheek portion (117) of the mask and situated in a 2 by 2 arrangement;
c) two or more sensors (104) embedded in the mask (102), wherein each sensor occupies one of the sensor positions;
d) a sensor node (108) disposed on a lateral flap (118) extending from the cheek portion (117) of the mask, wherein the sensor node (108) comprises a processing module and a transmitter (112); and
e) connecting leads (106) electrically coupling each of the two or more sensors (104) to the sensor node (108);
wherein when the flexible mask is applied to partially cover one side of the human's face, the sensor positions align with pain-related facial muscles in the human's face, wherein the sensors (104) are configured to detect biosignals from underlying facial muscles, wherein the processing module is configured to: (i) receive the biosignals from the plurality of sensors, (ii) analyze the biosignals to deduce facial expressions and monitor pain intensity levels experienced by the subject based on the deduced facial expressions, and (iii) transmit the pain intensity levels to a medical care provider, thus allowing the medical care provider to continually monitor the pain intensity levels experienced by the subject thereby providing effective and efficient pain management.

13. The system (100) of claim 12, wherein the flexible mask (102) is composed of polydimethyl silicone elastomer (PDMS).

14. The system (100) of claim 12, wherein the sensors (104) comprise Ag/AgCl electrodes.

15. The system (100) of claim 14, wherein the electrodes are formed on an inner surface of the mask (102) such that the electrodes are directly contacting skin when the mask is placed on the human's face.

16. The system (100) of claim 12, wherein the pain-related facial muscles are frontalis, corrugator, orbicularis oculi, levator, zygomaticus, and risorius.

17. The system (100) of claim 12 comprising two sensors (104), wherein a first sensor occupies a distal-most sensor position located on the forehead portion (116) of the mask, wherein a second sensor occupies a first row and first column of the 2 by 2 arrangement in the cheek portion (117) of the mask, wherein the first sensor detects biosignals from a corrugator facial muscle and the second sensor detects biosignals from a zygomatic facial muscle.

18. The system (100) of claim 12 comprising five sensors (104), wherein a first sensor and a second sensor occupy the two sensor positions on the forehead portion (116) of the mask, wherein a third sensor and a fourth sensor occupy the sensor positions at a first row of the 2 by 2 arrangement in the cheek portion (117) of the mask, wherein a fifth sensor occupies the sensor position at a second row and second column of the 2 by 2 arrangement, wherein the first sensor detects biosignals from a corrugator facial muscle, the second sensor detects biosignals from a frontalis facial muscle, the third sensor detects biosignals from a levator facial muscle, the fourth sensor detects biosignals from an orbicularis oculi facial muscle, and the fifth sensor detects biosignals from a zygomatic facial muscle.

19. The system (100) of claim 12, wherein the facial expressions comprise one or more of a smile, frown, and a wrinkle nose.

20. The system (100) of claim 12, wherein the biosignals comprise surface electromyogram (sEMG) signals.
